(19) United States
(12) Patent Application Publication
(10) Pub. No.: US 2004/0147500 A1
(43) Pub. Date: Jul. 29, 2004

(54) Injectable galenic formulation for use in a diagnosis or a photodynamic therapy and method for preparing same

(76) Inventors: Pierre- Herve Brun, Paris (FR); Alain Prudhomme, La Foret Fouesnant (FR); Jean-Luc Barnoux, Chaon (FR)

Correspondence Address: Clifford W Browning, Woodard Emhardt, Bank One Center Tower, 111 Monument Circle Suite 3700, Indianapolis, IN, 46204-5137, US

(21) Appl. No.: 10/484,154

(22) PCT Filed: Jul. 5, 2002
(86) PCT No.: PCT/IB02/02661

(30) Foreign Application Priority Data
Jul. 17, 2001 (EP) 01810708.6

(51) Int. Cl. A61K 31/555

(52) U.S. Cl. 514/185; 424/9.61

(57) ABSTRACT

The invention concerns an injectable galenic formulation for use in a diagnosis or a photodynamic therapy and the method for preparing same. The formulation contains: a compound of general formula (I), wherein: R₂ represents a H, OH or COOR₄ group, where R₄ is a hydrogen or an C₁-C₁₂ alkyl or a C₃-C₁₂ cycloalkyl; R₃ represents H, OH or a C₁-C₁₂ alkyl or alkoxy; and * represents an asymmetric carbon in the form of an alkali metal salt in an amount not exceeding 10 mg/ml, as photosensitizing agent, and a carrier in aqueous phase containing at least a benzyl alcohol-ethanol mixture or propylene glycol as solubilizing agent for the photosensitizing agent and a surfactant in an amount not exceeding 20 wt. % relative to the total weight of the formulation.

The present invention relates to an injectable galenic formulation for use in a diagnosis or in a photodynamic therapy (PDT), containing a photosensitising agent derived from a modified bacteriochlorophyll and a pharmaceutically acceptable carrier in an aqueous phase.

The present invention is also concerned with a method for preparing this injectable galenic formulation.

Over fifty years ago, it had been demonstrated that porphyrins were capable of accumulating in tumorous tissues and that, furthermore, they were capable of fluorescing in these tumorous tissues when irradiated, by absorbing light in situ.

Owing to their properties, these porphyrins have demonstrated that they were capable of providing an interesting means for detecting a tumour by localising fluorescence.

In recent years, the use of photosensitising agents in the treatment of cancer has been the object of numerous studies, which proved to be very promising, so that the interest in this technique, known as “photodynamic therapy” or PDT continues to increase.

In this technique of photodynamic therapy (PDT), a photosensitising agent is administered, which is capable of fixing itself preferentially in tissues in which the cells proliferate and said tissues are exposed to a light having a wavelength which is absorbed by the photosensitising product, from a monochromatic light source (laser) or a polychromatic light source (lamp).

The photosensitising product, which is not toxic per se, has the property, after having been excited by the absorption of photons, to revert to its basic state by transferring energy to ambient oxygen to form a singlet oxygen (type II photo-oxidation) and/or oxygenated free radicals (type I photo-oxidation).

These oxygen derivatives are extremely reactive and toxic and they oxidise all the tissue constituents with which they come in contact.

Furthermore their diffusion is very limited and the lifespan is short, so that the phototoxic effect induced by an exposure to light of the photosensitising agent remains localised.

The main indications for the photodynamic therapy are in cancerology, virology, ophthalmology, in particular for the treatment of senile macular degeneration (SMD), cardiology (restenosis after an angioplasty), rheumatology and dermatology.

A considerable number of photosensitising molecules have been studied for their use in photodynamic therapy.

The use of hematoporphyrin derivatives and, in particular, of Photofrin® (a sodium porfimer, which is a mixture of ethers and esters of dihematoporphyrin marketed by Quadra Logic Technologies, Inc. Vancouver, BC, Canada) in photodynamic therapy has largely been investigated (see for example Dougherty, T. J., Photochem. Photobiol., 46, 569 (1987); Kessel et al., Photochem. Photobiol. 46, 563 (1987); Dougherty, T. J., Semin. Surg. Oncol., 2, 24 (1986); McCaughan, J. S., Photochem. Photobiol. 46, 903 (1987); and Gomer, C. J., Semin. Hematol., 26 (1) 27 (1989)).

In most clinical studies, use was made of a hematoporphyrin derivative (HPD) which was comprised of a mixture of porphyrin compounds (monomers of porphyrin, hematoporphyrin, hydroxy-ethyl-vinyl-deuteroporphyrin, protoporphyrin and dimers/polymers of porphyrin) (see for instance, Daugherty, T. J. Photochem. Photobiol. 46, 569 (1987).

As a result of these studies, it was found that the therapeutic utilisation of these derivatives of hematoporphyrin suffered two major drawbacks.

The first one is that, at the wavelength of the light which activates the compound, the light does not penetrate sufficiently deeply into the tissue (see for instance, Doiron et al., pp. 281-291 in: Porphyrins in Tumor Phototherapy, Edited by A. Andreoni and R. Cuulbreddu, Plenum Press, New York, 1984), so that the use of these compounds is limited to the treatment of tumours of a small size and is inadequate for treating tumours located deep in the tissue.

The second major drawback of these derivatives of hematoporphyrin is that they tend to accumulate in the tissues of the patient, causing, in particular, a cutaneous and an ocular photosensitisation, which can persist over four to eight weeks after the treatment, and this requires that special precautions be taken, such as, for example, that exposure to sunlight be avoided, a cream containing a sunblocker be applied on the skin and that appropriate clothing and sunglasses be worn.

The only case of a hematoporphyrin derivative, which has been granted a marketing authorisation (MA) for its use in photodynamic therapy is that of Photofrin® (an MA has been granted in the USA in 1994 and in Europe in 1996), an injectable aqueous solution containing a mixture of porphyrin compounds absorbing light at 630 nm and producing a cutaneous and an ocular photosensitisation, which persists even two months after the administration.

To overcome the drawbacks of the hematoporphyrin derivatives indicated hereabove, a new generation of photosensitising agents has been developed, having as a common feature that they are pure compounds having a well-defined structure and absorbing light at a wavelength situated in the range from 650 to 800 nm.

As examples of photosensitising agents used in photodynamic therapy and belonging to this second generation, one can mention the derivatives of benzoporphyrin (see, for example: A. M. Richter, S. Yip, H. Meadows et al. “Photosensitizing potencies of the structural analogues of benzoporphyrin derivative in different biological test systems” J Clin Laser Med Surg 1996,14, 335-341; J. G. Levy, A. Chan, H. A. Strong “The clinical status of benzoporphyrin derivative” SPIE 1996, 2625: 86-95; U. Schmidt-Erfurht, W. Bauman, E. Gragoudas et al. “Photodynamic therapy of experimental choroidal melanoma using lipoprotein-delivered benzoporphyrin” Ophthalmology 1994,101: 89-99).

The benzoporphyrin derivatives offer the advantage that these compounds are activated by a light having a wavelength (≈690 nm) higher than that activating hematoporphyrin derivatives, so that the light penetrates more deeply into the tissues.

Furthermore, the use of these derivatives of benzoporphyrin has shown that their cutaneous and ocular photosensitising effect was considerably decreased.

However, benzoporphyrin derivatives (BPD) suffer the major drawback that they are insoluble in aqueous media, so that their pharmaceutical utilisation by parenteral administration has proven difficult.

U.S. Pat. No. 5,289,378 proposes to remedy this drawback by incorporating the benzoporphyrin derivatives into liposomes.

As an example of an injectable liposome formulation containing a benzoporphyrin derivative for use in photodynamic therapy for treating predominantly visible central neovascular forms of senile macular degeneration (SMD), one can mention Visudyne® (Ciba-Vision) which contains as the photosensitising molecule, the compound verteporfin, which absorbs light at a wavelength of 690 nm and causes a cutaneous and an ocular photosensitisation which is limited in time.

As other examples of photosensitising agents useful in photodynamic therapy, belonging to this new generation, one can mention, in particular, the bacteriochlorophyll derivatives of the general following formula:

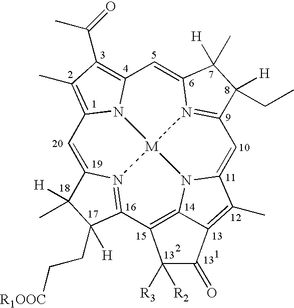

In which M represents an atom of a metal or 2 atoms of hydrogen and R₁, R₂ and R₃ represent, each one independently, an atom of hydrogen or a variable group.

These compounds are termed, for example:

Bchl or bacteriochlorophyll when:

M represents Mg

R₁ represents a phytyl group

R₂ represents an atom of hydrogen

R₃ represents a —COOCH₃ group;

Bchlide or bacteriochlorophyllide when

M represents Mg

R₁ represents an atom of hydrogen

R₂ represents an atom of hydrogen

R₃ represents a group —COOCH₃; and

Bpheide or bacteriopheophorbide when:

M represents 2 atoms of hydrogen and

R₁ represents an atom of hydrogen

R₂ represents an atom of hydrogen

R₃ represents a —COOCH₃ group.

Some of these derivatives are described, in particular, in DE-A-41 21 876, EP-A0584 552 and WO 97/19081.

Other bacteriochlorophyll derivatives, which have proven to be particularly interesting are derivatives of bacteriochlorophyll in which the atom of magnesium is replaced by an atom of palladium, as described, for example, in WO 00/33833.

Amongst the bacteriochlorophyll derivatives modified at the metal and described in WO 00/33833, the compounds having the following general formula (I):

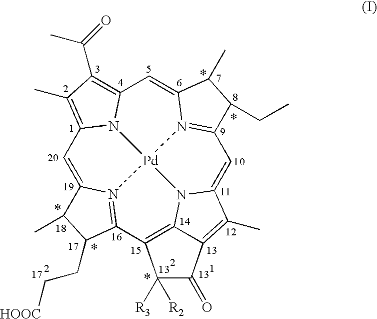

in which

R₂ represents a group H, OH or COOR₄, wherein R₄ is a hydrogen or a C₁-C₁₂ alkyl or a C₃-C₁₂ cycloalkyl,

R₃ represents H, OH or a C₁-C₁₂ alkyl or alcoxy and

* represents an asymmetrical carbon,

or the salts thereof, have proven to be of a particular interest and, among these compounds of Formula (I) hereabove, the Pd-bacteriopheophorbide a having the following Formula (II):

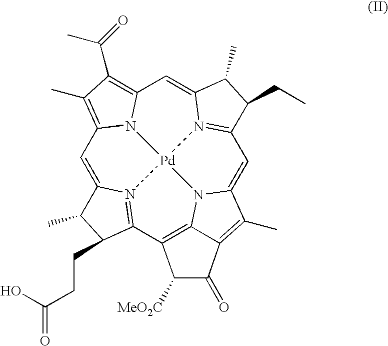

or a salt thereof, has proven to have a phototoxicity and a stability in the course of time much greater than those of the other derivatives of bacteriochlorophyll.

Furthermore the Pd-bacteriopheophorbide a, also called Pd-Bpheide a, absorbs light at a wavelength of about 760 nm, i. e. at a wavelength higher than that of the other photosensitising agent, which allows a deeper penetration of the light into the tissues and it causes no or little cutaneous photosensitisation.

However, all the compounds having the Formula (I) hereabove, have strongly marked lipophilic character with a very strong tendency to form aggregates in an aqueous medium, which makes their pharmaceutical use difficult by parenteral administration.

WO 00/33833 claims, in particular, a pharmaceutical composition including a compound of the Formula (I) hereabove, preferably, the Pd-Bpheide a of Formula (II) hereabove, and a pharmaceutically acceptable carrier.

In the description of WO 00/33833, it is however mentioned that, for preparing such a pharmaceutical composition, which can be administered by the systemic route, it is necessary to add an amount of a non-toxic surface-active agent sufficient for solubilizing the compound.

Compositions containing the Pd-Bpheide a, described specifically in WO 00/33833 and used for in vivo tests in animals, contain a very high amount of Cremophore® EL (20-50%) which has proven necessary for solubilizing the photosensitising agent.

In these compositions, Cremophore® EL functions as a surface-active agent capable of decreasing the formation of aggregates and of solubilizing the Pd-Bpheide a.

Cremophore® EL is an emulsifier excipient of the polyoxyethylenated castor oil type, which is sold commercially and which is known for its capacity to solubilize a broad range of lipophilic active principles in pharmaceutical compositions.

However, a composition containing more than 5% Cremophore® EL is difficult to administer by the systemic route in humans, because of the known risk of causing undesirable effects, such as allergy.

Trials aimed at replacing Cremophore® El by other surface-active agents known for their use as solubilizing agents for lipophilic principles in injectable solutions, such as Solutol HS 15™, propylene glycol, polysorbate 80, soy lecithins, egg yolk lecithins and N-methyl-pyrrolidone (Pharmasolve®), have not produced satisfactory results.

Because of the particularly interesting properties of the compounds of Formula (I) hereabove and in particular those of Pd-Bpheide a of the Formula (ii) hereabove as photosensitising agents for photodynamic therapy, it is necessary to provide a galenic formulation containing a compound of Formula (I) hereabove which can be administered to humans by the intravenous route, without the risk of complications due to an excipient.

The objective of the present invention is to provide an injectable galenic formulation in which the compound of Formula (I) hereabove is completely solubilized and which contains a strongly reduced amount of surfactant.

This objective was attained when the inventors discovered that, surprisingly, the addition of a small amount of non aqueous co-solvents and, in particular, the addition of a small amount of a mixture of benzyl alcohol/ethanol or of propylene glycol to the Pd-Bpheide a, makes it possible to solubilize the Pd-Bpheide a in an aqueous solution after its conversion into a salt.

According to a first aspect, the present invention is concerned with an injectable galenic formulation for use in a diagnosis or in a photodynamic therapy (PDT), said galenic formulation containing

a compound represented by the following general formula (I):

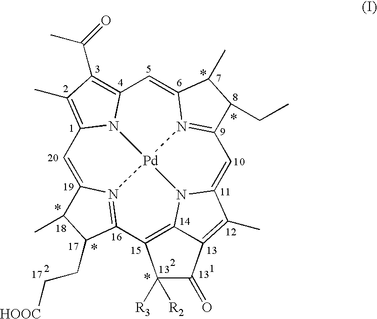

in which

R₂ represents a group H, OH or COOR₄, wherein R₄ is a hydrogen or a C₁-C₁₂ alkyl or a C₃-C₁₂ cycloalkyl,

R₃ represents H, OH or a C₁-C₁₂ alkyl or alcoxy and

* represents an asymmetric carbon,

in the form of a salt of an alkaline metal, in an amount not exceeding 10 mg/ml, as the photosensitising agent, and

a pharmaceutically acceptable carrier in an aqueous phase, characterised in that the pharmaceutically acceptable carrier in an aqueous phase contains, at least, a mixture of benzyl alcohol—ethanol in a ratio from 15:85 to 25:75 or propylene glycol, acting as a solubilizing agent for the photosensitising agent and a surfactant capable of decreasing the aggregation of the photosensitising agent, in an, amount not exceeding 20 wt. % relative to the total weight of the formulation.

According to a second aspect, the present invention is concerned with a method for the preparation of an injectable galenic formulation for use in a diagnosis or in a photodynamic therapy (PDT) containing a compound represented by the following Formula (I):

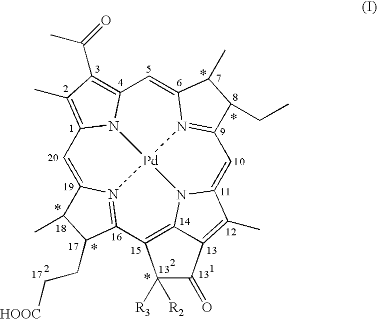

in which

R₂ represents a group H, OH or COOR₄, wherein R₄ is a hydrogen or a C₁-C₁₂ alkyl or a C₃-C₁₂ cycloalkyl,

R₃ represents H, OH or a C₁-C₁₂ alkyl or alcoxy and

* represents an asymmetric carbon,

in the form of a salt of an alkaline metal, in an amount of up to 10 mg/ml, as the photosensitising agent and a pharmaceutically acceptable carrier in an aqueous phase, said method including, in the order indicated, the steps consisting in

(1) wetting the photosensitising agent of Formula (I) in its acid form with a solubilizing agent comprised of a mixture of benzyl alcohol—ethanol in a ratio from 15:85 to 25:75, or of propylene glycol;

(2) adding a surface-active agent in an amount not exceeding 20 wt. % relative to the final weight of the formulation;

(3) adding a hydroxide of an alkaline metal in an amount sufficient for neutralising the acid function of the photosensitising agent of Formula (I)

(4) stirring the mixture in the aqueous phase sufficiently to ensure a complete conversion of the photosensitising agent into the salt thereof and its complete solubilization

(5) adding a buffering agent designed for adjusting the pH to a physiologically acceptable value of 7.2±0.4;

(6) if necessary, adjusting the volume of the formulation with water for injectable preparations.

According to a third aspect, the present invention is concerned with an injectable galenic formulation obtained by this method.

The galenic formulation of the present invention contains a strongly reduced amount of surfactant, which functions essentially to decrease the formation of aggregates.

Accordingly, the present invention provides a galenic formulation in which the photosensitising agent, namely a compound of Formula (I) hereabove, is completely solubilized in the form of a salt and which can be injected with minimal risks to humans in the context of a photodynamic therapy.

Other advantages of the present invention will become apparent from the following detailed description of the invention.

### DETAILED DESCRIPTION OF THE INVENTION

In accordance with the present invention, the injectable galenic formulation for use in a photodynamic therapy contains:

a compound of Formula (I)

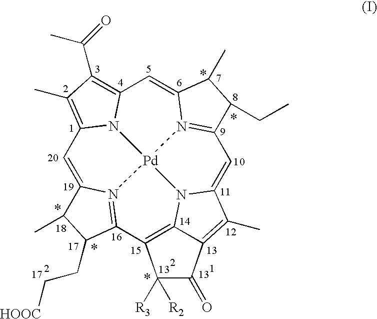

in which

R₂ represents a group H, OH or COOR₄, wherein R₄ is a hydrogen or a C₁-C₁₂ alkyl or a C₃-C₁₂ cycloalkyl,

R₃ represents, H, OH or a C₁-C₁₂ alkyl or alcoxy and

* represents an asymmetric carbon,

in the form of a salt of an alkaline metal, as the photosensitising agent and

a pharmaceutically acceptable carrier in an aqueous phase.

In the galenic formulation of the present invention, the compound of Formula (I) hereabove is, preferably, the Pd-Bpheide a of the Formula (II) hereafter:

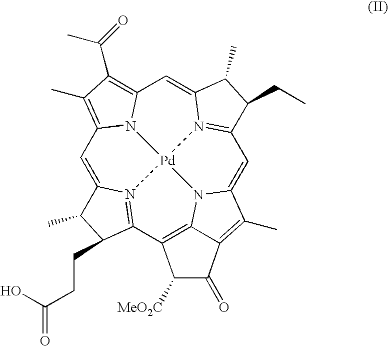

in the form of a salt of an alkaline metal.

The alkaline metal salt of the compound of Formula (I) or of the compound of Formula (II) can be a sodium salt or a potassium salt.

Preferably, the alkaline metal salt of the compound of Formula (I) or of the compound of Formula (II) is a sodium salt.

The compounds of the Formula (I) hereabove and, in particular, the Pd-Bpheide a, represented by Formula (II) hereabove, as well as their alkaline metal salts, can be prepared as described in WO 00/33833.

The amount of photosensitising compound contained in the injectable galenic formulation of the present invention should not exceed 10 mg/ml, because, when the amount is higher than 10 mg/ml, the photosensitising compound is not completely solubilized.

When the injectable galenic formulation is designed for a utilisation in the context of a photodynamic therapy, the amount of photosensitising agent contained in the formulation should not be, preferably, lesser than 0.1 mg/ml, because below this concentration, the volume of the formulation to be administered would be too important.

However, amounts of photosensitising agent lesser than 0.1 mg/ml can be envisaged when the injectable galenic formulation of the present invention is intended for a utilisation in the context of a photodynamic diagnosis.

The pharmaceutically acceptable carrier in an aqueous phase contained in the injectable galenic formulation of the present invention is designed for solubilizing the photosensitising agent while ensuring the long-term stability of the formulation.

This pharmaceutically acceptable carrier in an aqueous phase contains, at least, a mixture of benzyl alcohol—ethanol or propylene glycol as the solubilizing agent for the photosensitising agent and a surface-active agent which makes it possible to decrease the aggregation of the photosensitising agent, in an amount not exceeding 20 wt. % relative to the total weight of the formulation.

When the pharmaceutical carrier contains a mixture of benzyl alcohol—ethanol as the solubilizing agent, the ratio of the benzyl alcohol:ethanol mixture should be from 15:85to25:75vol./vol.

When the mixture contains less than 15 parts of benzyl alcohol, the photosensitising agent is not completely solubilized

When the mixture contains more than 25 parts of benzyl alcohol, the mixture is no longer miscible with water.

In a particularly preferred version of the invention, the carrier contains a mixture benzyl alcohol—ethanol in a ratio benzyl alcohol : ethanol of 20:80.

The benzyl alcohol—ethanol mixture is preferably contained in the carrier in an amount not exceeding 5 vol. % relative to the total volume of the formulation.

The benzyl alcohol and the ethanol contained in the injectable galenic formulation of the present invention must satisfy the purity requirements set out for injectable formulations, in particular in the European Pharmacopoeia and the US pharmacopoeia (USP).

When the pharmaceutical carrier contains propylene glycol as the wetting agent, the propylene glycol is preferably contained in the carrier in an amount not exceeding 5 vol. % relative to the total volume of the formulation.

The propylene glycol contained in the injectable galenic formulation of the present invention must satisfy the purity requirements set out for injectable formulations, in particular in the European Pharmacopoeia and the US Pharmacopoeia (USP).

The surface-active agent contained in the pharmaceutically acceptable carrier of the aqueous phase can be any surface-active agent having the capacity of decreasing the aggregation of the photosensitising agent in the injectable galenic formulation and it should be contained therein in an amount not exceeding 20 wt. % relative to the total weight of the formulation.

This surface-active agent can be, for example, Cremophore® EL (BASF, Germany), Cremophore® EL P (BASF, Germany), Solutol HS 15™ (BASF, Germany), propylene glycol, polysorbate 80, soy lecithins, egg yolk lecithins and N-methyl-pyrrolidone (Pharmasolve®, BASF, Germany).

Preferably, the surface-active agent is Cremophore® EL, Cremophore® EL P or Solutol HS 15™.

A particularly preferred formulation of the present invention contains, as the surface-active agent, Cremophore® EL, Cremophore® EL P in an amount not exceeding 5 vol. % relative to the total volume of the formulation.

In a particularly preferred version of the present invention, the formulation contains 0.25% wt./wt. of Pd-Bpheide a, 5% vol./wt. of a mixture of benzyl alcohol—ethanol having a ratio benzyl alcohol:ethanol of 20:80 and 5% wt./wt. of Cremophore® EL P.

The injectable galenic formulation of the present invention can be prepared according to a method comprising, in the order indicated, the steps consisting in:

(1) wetting the photosensitising agent of Formula (I) in its acid form or of Formula (II) in its acid form with a solubilizing agent comprised of a mixture benzyl alcohol—ethanol in a ratio of 15:85 to 25:75 or of propylene glycol;

(2) adding a surface-active agent in an amount not exceeding 20 wt. % relative to the final weight of the formulation;

(3) adding a hydroxide of an alkaline metal in an aqueous solution in an amount sufficient for neutralising the photosensitising agent of Formula (I) or of Formula (II);

(4) stirring the mixture in the aqueous phase sufficiently to ensure the complete conversion of the photosensitising agent into its salt and its complete solubilization;

(5) adding a buffering agent for adjusting the pH to a physiologically acceptable value of 7.2±0.4;

(6) if necessary, adjusting the volume of the formulation with water suitable for injectable preparations.

In a preferred embodiment of the invention, the solubilizing agent is preferably added in an amount not exceeding 5 vol. % relative to the volume of the total formulation.

In the method according to the present invention, the solubilizing agent added to wet the photosensitising agent is preferably a mixture of benzyl alcohol—ethanol in a ratio 20:80.

The wetting with pure benzyl alcohol is not possible, owing to the non miscibility of benzyl alcohol with the diluted sodium hydroxide solution.

The addition of ethanol to the benzyl alcohol in the proportions indicated ensures the miscibility of the organic wetting phase with the diluted sodium hydroxide solution.

The benzyl alcohol and the ethanol or the propylene glycol added to wet the photosensitising agent must satisfy the purity requirements for their use in injectable formulations as set out in the European Pharmacopoeia and the US Pharmacopoeia (USP).

The surface-active agent added to decrease the aggregation of the photosensitising agent in the injectable galenic formulation is, for example Cremophore® EL, Cremophore® EL P, Solutol HS 15™, propylene glycol, polysorbate 80, soy lecithins, egg yolk lecithins and N-methyl-pyrrolidone (Pharmasolve®).

Preferably, the surface-active agent added is Cremophore® EL, Cremophore® EL P or Solutol HS 15™.

The amount of surface-active agent, which is added, will vary depending upon the surface-active agent used and on the final concentration of the photosensitising agent in the formulation.

This amount will, however, not be in excess of 20 wt. % relative to the final weight of the formulation.

In a particularly preferred version of the method of the present invention, the surface-active agent added is Cremophore® EL or Cremophore® EL P, in an amount not exceeding 5 vol. % relative to the total volume of the formulation.

The hydroxide of an alkaline metal used in the method of the present invention can be sodium hydroxide or potassium hydroxide and is added in the form of an aqueous solution.

The hydroxide of an alkaline metal is added to the compound of Formula (I) in its acid form or to the compound of Formula (II) in its acid form, in an amount sufficient for converting totally the acid function of the compound of Formula (I) or of the compound of Formula (II) into the salt thereof.

Preferably, the hydroxide of an alkaline metal used is sodium hydroxide, so that the injectable galenic formulation obtained contains the compound of Formula (I) and, preferably, the compound of Formula (II) in the form of the sodium salt thereof.

Sufficient stirring should be ensured for enabling a complete conversion of the photosensitising agent into its salt and the total solubilization of the salt in the carrier.

The agent used for adjusting the pH in the method of the present invention can be, for example citric acid, phosphoric acid and its sodium salts (monobasic and dibasic), D-tartaric acid, L-tartaric acid, D-malic acid and L-malic acid.

The agent used for adjusting the pH is added in the amount necessary for buffering the injectable formulation to a pH physiologically acceptable of 7.2±0.4.

In a particularly preferred embodiment of the present invention, the agent used for adjusting the pH is citric acid, because it confers to the formulation a stability in the course of time and in the presence of air, which is improved.

In order to improve the stability of the injectable formulation obtained by the method of the present invention, the different steps (1) to (6) of the method will be carried out under an atmosphere of an inert gas.

In the injectable galenic formulation obtained by the method of the present invention, the compound of Formula (I) or, preferably, the compound of Formula (II) is completely solubilized, which is demonstrated by the fact that the formulation is clear and devoid of any precipitate.

Furthermore, the injectable formulation obtained by the method of the present invention exhibits an excellent stability in the course of time.

In the galenic composition of the present invention, the compound of Formula (I) and, preferably, the compound of Formula (II), is present in the form of monomers with, however, a certain quantity of the compound, forming aggregates (oligomers).

However, upon injection of the injectable galenic formulation of present invention, the oligomers forms present in the formulation are transformed rapidly into monomers when they come in contact with a physiological liquid, in particular with blood, so that the active photosensitising product is entirely converted into monomers.

The analysis of the injectable formulation of the present invention by UV spectroscopy can provide interesting indications on the Pd-Bpheide a contained in the formulation, since the Pd-Bpheide a exhibits a peak in the vicinity of 760 nm, the aggregation thereof can be determined from the occurrence of a neighbouring peak at about 815 nm and the degradation thereof, in particular through oxidation, can be inferred from the hypsochromic displacements of the maximums, in particular of that in the vicinity of 760 nm.

The following examples are given for illustrating the invention. However, they should in no case be considered as limiting the scope of the present invention.

### EXAMPLES

Unless specified otherwise, all the reactants used in the examples hereafter are reactants which are available commercially, in particular from SDS (BP 4-Valdonne, F-13124 Peypin), Merck Eurolab (Europarc, F-33608 Pessac Cedex), BASF (Germany), Flüka (Buchs, Switzerland) etc.

### Preparation of the Photosensitising Agent Pd-Bpheide a

Note: the entire preparation was carried out under inactinic light and under an atmosphere of nitrogen.

### 1) Extraction of the bacteriochlorophyll a (BChla)

Reactants used:

| | | |
|---|---|---|
| Rhodovolum Sulfidophilum cells | 11 | kg |
| Methanol [SDS or Merck, pure for synthesis, purity ≧ 99.8%] | 126 | l |

Method used:

Freeze-dried Rhodovolum Sulfidophilum cells (11 kg) were extracted with methanol (57 l) at 25° C. and the medium was filtered. The cell residue was suspended again in 47 l of methanol and filtered. After rinsing the residue with 22 l of methanol, the filtrates were pooled together and concentrated under reduced pressure.

Approximately 3500 g of a crude product containing 70 g of BChla (amount of BChla determined by UV analysis).

### 2) Synthesis of the bacteriopheophorbide a

Reactants used:

| | | |
|---|---|---|
| Crude product containing 70 g of bacteriochlorophyll a | 3500 | g |
| obtained at step 1) | | |
| Trifluoroacetic acid [SDS, pure for syntheses, purity ≧ 99%] | 7 | l |
| Chloroform [SDS, pure for syntheses, purity ≧ 99.9%] | 11.2 | l |

Method used:

The crude product containing 70 g of bacteriochlorophyll a (3500 g) was solubilized in chloroform (11.2 l) and trifluoroacetic acid (7 l ) was added. The progression of the reaction was monitored by TLC. When no initial product was detected any more, 56 l of water and 14 l of chloroform were added to the reaction medium. After stirring the mixture and allowing it to settle, the two phases were separated. The organic phase was washed with water (25 l) and the chloroform was concentrated under reduced pressure to a volume of 6 l.

The crude Bpheide was obtained as a solution in the chloroform and was not isolated before its purification.

### 3) Purification of the bacteriopheophorbide a

Reactants used:

Crude Bpheide solution in chloroform, obtained at step 2)

| | | |
|---|---|---|
| Cyclohexane [SDS, pure for syntheses, purity ≧ 99%] | 10 | l |
| Pentane [SDS, pure for syntheses, purity ≧ 99%] | 7 | l |
| Methanol [SDS or Merck, pure for syntheses, | 3 | l |
| purity ≧ 99.8%] | | |
| Silica [SDS, 60 A C.C., 70-200 μm] | 300 | g |
| Ethyl acetate [Merck, pure, purity 99.5%] | 6 | l |

Method used:

The cyclohexane (10 l) was added to the crude solution of Bpheide in chloroform and the solvents were distilled under reduced pressure, with addition of cyclohexane until the chloroform was eliminated and a volume of 7 l was reached. The product was then precipitated through the addition of pentane (7 l), stirred for 15 minutes at 30° C. and filtered. After drying in a vacuum oven (30° C.) 60 g of a semi-purified product were obtained (UV purity: 50-60%).

The semi-purified product (60 g) was dissolved in a mixture chloroform/methanol 95/5 (6 l) and 300 g of silica were added. After stirring 10 minutes, the medium was filtered. The filtrate was diluted with 6 l of ethyl acetate and the solvents were distilled under reduced pressure. As the concentration progressed, ethyl acetate was added until obtaining a distillate having a specific gravity of 0.9. The volume of the solution was then adjusted to 3.0 l. The solution was filtered and the powder obtained was dried in an oven under vacuum at 30° C.

Approximately 35 g of purified Bpheide were obtained (UV purity: 80%).

### 4) Synthesis of palladium-bacteriopheophorbide a

Reactants used:

| | | |
|---|---|---|
| Crude Bpheide solution in chloroform, obtained at step 3) | 6.25 | g |
| Palladium acetate [SDS or MERCK, content ≧ 99 wt./wt.] | 13.15 | g |
| Ascorbic acid [SDS, purity ≧ 99%] | 105 | g |
| Chloroform [SDS, pure for syntheses, purity ≧ 99.9%] | 2.2 | l |
| Methanol [SDS, pure for analyses, purity ≧ 99.8%] | 3.3 | l |
| Sodium sulphate, anhydrous [SDS for analyses, | 1.5 | kg |
| purity ≧ 99.5%] | | |

Method used:

Ascorbic acid (105 g) was dissolved in 3.3 l of methanol and the solution was heated to 35° C. The Bpheide (6.25 g) solubilized in 1.5 l of chloroform was added in 10 minutes to the above solution. After stirring for 10 minutes, the palladium acetate (13.15 g) solubilized in 0.7 l of chloroform was added in 10 minutes to the reaction medium. The progression of the reaction was monitored by UV analysis. After 15 minutes, the mixture was cooled down to 20° C. and poured into 20 l of water. After stirring the mixture and allowing it to settle, the phases were separated. The organic phase was washed with water (20 l) until a pH≧5 was reached and then dried over sodium sulphate (1.5 kg). After concentration of the solvents under reduced pressure, 5.85 g of the crude product were obtained (UV purity: 85%).

### 5) Purification of palladium-bacteriopheophorebide a

Reactants used:

| Crude Pd-Bpheide obtained at step 4) | 5.85 | g |
|---|---|---|
| | (UV purity: 85%) | |
| Chloroform [SDS, pure for syntheses, purity ≧ 99.9%] | 175 | ml |

Method used:

The crude product (5.85 g) was taken up in chloroform (175 ml) and stirred for 5 minutes. The suspension was filtered and the solids obtained were dried under vacuum in an oven (30° C.).

5.0 g of purified Pd-Bpheide a were isolated (HPLC purity>97%).

UV/VIS in chloroform: λₘₐₓ [nm], relative intensity at 330 (0.798); 385 (0.597); 535 (0.292); 760 (1.711).

NMR-¹H in D₆-acetone [ppm]: 9.27, 8.86, 8.77 (each s, 1H, 5-, 10-, 20-H); 6.01 (s, 1H, 13²-H) ; 4.7 to 4.0 (4 m, 1H, 7-, 8-, 17-, 18-H ; 3.86 (s, 3H, 13²-CO₂CH₃); 3.51 (s, 3H, 2-CH₃); 3.34 (s, 3H, 12-CH₃); 3.07 (s, 3H, 3-COCH₃); 2.7 to 2.1 (3m, 2H, 8-, 17¹-, 17²-CH₂); 1.79, 1.69 (each d, 3H, 7-, 18-CH₃); 1.09 (t, 3H, 8-CH₂CH₃) MS calculated for C₃₅H₃₆N₄O₆Pd: 714.1670 (M⁺). Found: 714.1688 (M⁺).

Elemental analysis:

| | | | | | |
|---|---|---|---|---|---|
| Calculated %: | C: 58.79 | H: 5.07 | N: 7.83 | O: 13.42 | Pd: 14.88 |
| Found (%): | C: 59.13 | H: 5.12 | N: 7.72 | O: 13.33 | Pd: 14.69 |

The Pd-Bpheide a obtained hereabove was used for preparing the injectable formulations hereafter.

Injectable Formulation 1

Formulation 1 containing 2.5 mg/ml of Pb-Bpheide a as the photosensitising agent

Carrier:

Organic phase: benzyl alcohol/ethanol (20/80) 5% vol. wt.

Surfactant: Cremophore® EL P 5% wt./wt.

NaOH: 0.028 M

pH adjusted to 7.2±0.4 with citric acid 0.01 M

Formulation 1 was prepared as follows:

In an appropriately sized glass container containing 2.5 mg of Pd-Bpheide a prepared as describe above, 50 μl of a 20:80 vol./vol. mixture of benzyl alcohol and of ethanol were added and the mixture was stirred moderately. Then, 50 mg of Cremophore® EL P were added to the mixture and the mixture was stirred with a “Vortex”. Thereafter, 500 μl of a sodium hydroxide solution NaOH 8.4×10⁻³ M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations (WIPP).

The exact composition of Formulation 1 is given in Table 1 hereafter:

TABLE 1

| Name of | Formula | | | Relevant |
|---|---|---|---|---|
| component | Per unit dose | Percentage (%) | Function | standards |

Photosensitising agent

| | | | |
|---|---|---|---|
| Pd-Bpheide a | 2.5 mg | 0.25% wt./wt. | AP |

Other components

| | | | | |
|---|---|---|---|---|
| Alcohol, absolute | 40 μl | 4% vol./wt. | Co-solvent | EP 4^{th} edition 1318, A2000 |
| Benzyl alcohol | 10 μl | 1% vol./wt. | Co-solvent | EP 4^{th} edition 0256, 1997 |
| Cremophore ® | 50 mg | 5% wt./wt. | Surfactant | EP 4^{th} edition 1082, 1997 |
| EL P | | | | |
| NaOH | 500 μl | 50% vol./wt. | Neutralising | EP 4^{th} edition 0677, 1997 |
| 8.4 × 10⁻³ M | | | agent | |
| Citric acid, | QS to | QS to | Buffering | EP 4^{th} edition 0456, 1997 |
| 0.01 M | 7.2 ± 0.4 | 7.2 ± 0.4 | agent | |
| WIPP | QS to 1 g | QS to 100 | Diluent | EP 4^{th} edition 0169, A2000 |

This Formulation 1 of the present invention is clear, but strongly coloured (dark purple) and it does not contain any trace of a precipitate, which indicates that the Pd-Bpheide a is completely solubilized.

The UV absorption spectrum of the Pd-Bpheide a in this Formulation 1 of the present invention exhibited 5 main peaks at 344, 385, 538, 763 and 818 nm, with a ratio OD 763/818 in excess of 1, indicating that the monomer determined spectrally amounted to more than 50% of the product.

Under the ICH conditions, this Formulation 1 is stable at 4° C. during at least 6 months, as verified by HPLC and UV analyses.

Toxicity studies were carried out on the injectable Formulation 1 administered intravenously as a single injection or as repeated injections, to different animal species:

Male rats (Sprague Dawley): 6 to 12 animals for each dose (2×5, 2×10 and 2×20 mg/kg. The product was injected intravenously into the caudal vein.

Rabbits (New-Zealand albino rabbits): 5 animals received the dose of 5 mg/kg. The product was injected three times intravenously, once intra-arterially and once perivenously.

Monkeys (Cynomolgus, of a weight varying from 2.5 to 3.2 kg): 6 to 10 animals of each sex for each dose (1, 2, 5 and 6 mg/kg). The product was injected intravenously into the saphenous vein or into the cephalic vein, once each day during 14 days.

Monkeys (Cyonomolgus of a weight varying from 2.25 to 4.7 kg): 3 to 6 animals for each dose (2 and 5 mg/kg). The product was injected intravenously into the saphenous vein or into the cephalic vein.

The assessment of the animals was made on the basis of clinical signs, biological analyses carried out on the blood and on the urine, cardiovascular examinations and of histopathological examinations.

These studies showed that the Pd-Bpheide a and its injectable Formulation 1 were well tolerated at injection speeds up to 4 ml/min.

Injectable Formulation 2

Formulation 2 containing 5 mg/ml of Pd-Bpheide a as photosensitising agent

Carrier:

Organic phase: benzyl alcohol/ethanol (20/80), 5% vol./wt.

Surfactant: Cremophore® EL 5% wt./wt.

NaOH: 0.028 M

pH adjusted to 7.2±0.4 with citric acid 0.01 M

Formulation 2 was prepared as follows:

In an appropriately sized glass container containing 5 mg of Pd-Bpheide a prepared as describe above, 50 μl of a 20:80 vol./vol. mixture of benzyl alcohol and of ethanol were added and the mixture was stirred moderately. Then, 50 mg of Cremophore® EL were added to the mixture and the mixture was stirred with a “Vortex”. Thereafter, 500 μl of a sodium hydroxide solution NaOH 0.028 M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations (WIPP).

The exact composition of Formulation 2 is given in Table 2 hereafter:

TABLE 2

| Name of | Formula | | | Relevant |
|---|---|---|---|---|
| component | Per unit dose | Percentage (%) | Function | standards |

Photosensitising agent

| | | | |
|---|---|---|---|
| Pd-Bpheide a | 5 mg | 0.5% wt./wt. | AP |

Other components

| | | | | |
|---|---|---|---|---|
| Alcohol, absolute | 40 μl | 4% vol./wt. | Co-solvent | EP 4^{th} edition 1318, A2000 |
| Benzyl alcohol | 10 μl | 1% vol./wt. | Co-solvent | EP 4^{th} edition 0256, 1997 |
| Cremophore ® | 50 mg | 5% wt./wt. | Surfactant | EP 4^{th} edition 1082, 1997 |
| EL | | | | |
| NaOH 0.028 M | 500 μl | 50% vol./wt. | Neutralising | EP 4^{th} edition 0677, 1997 |
| | | | agent | |
| Citric acid, | QS to | QS to | Buffering | EP 4^{th} edition 0456, 1997 |
| 0.01 M | 7.2 ± 0.4 | 7.2 ± 0.4 | agent | |
| WIPP | QS to 1 g | QS to 100 | Diluent | EP 4^{th} edition 0169, A2000 |

This Formulation 2 of the present invention is clear but strongly coloured (dark purple) and it does not contain any trace of a precipitate, which indicates that the Pd-Bpheide a is completely solubilized.

The UV absorption spectrum of the Pd-Bpheide a in this Formulation 2 of the present invention exhibited 5 main peaks at 344, 385, 538, 763 and 818 nm with a ratio OD 763/818 close to 1, indicating that the monomer determined spectrally amounted to 50% of the product.

Under the ICH conditions, this Formulation 2 is stable at 4° C. during at least 6 months, as verified by HPLC and UV analyses.

The following biological tests have made it possible to demonstrate the important role and the need for a good solubilization of the active principle, as well as for a reduction of the aggregated form into the monomer form.

An investigation of the disaggregation of the formulated product, once injected, was carried out with New-Zealand white rabbits, weighing 3 to 4 kg.

Three rabbits were used in this investigation.

The rabbits received a dose of 5 mg/kg into the auricular vein, namely 3 ml of the injectable Formulation 1 containing 5 mg/ml to a 3 kg rabbit.

The speed of infusion varied from 0.3 to 1 ml/min.

The absorption spectrum of the Pd-Bpheide a was measured continuously by spectral reflectance, using a multiple-channel detection system. This system makes possible the detection of the product in the tissues of the living animal and enables a monitoring of the evolution of the aggregates in the organism.

Under the conditions indicated, the disaggregation in vivo of the oligomers to produce monomers proceeds very rapidly and requires from 15 to 30 minutes.

In the absence of Cremophore in the formulation, no disaggregation could be obtained in vivo and the concentration of the monomer produced remained very low.

Toxicity studies were carried out on the injectable Formulation 2 administered intravenously as a single injection or as repeated injections, to different animal species:

1—Mice (OF, of a weight varying from 25 to 35 grams): 10 animals of each sex for each dose (2×25, 2×50, 2×75 and 1×100 mg/kg).The product was injected into the caudal vein.

2—Rats (Sprague Dawley, of a weight varying from 150 to 230 grams): 4 to 10 animals of each sex for each dose (from 4 to 200 mg/kg). The product was injected into the caudal vein.

3—Monkeys (Cynomolgus of a weight varying from 1.8 kg to 3.5 kg): 1 animal of each sex for each dose (8, 16 and 24 mg/kg). The product was injected into the saphenous vein or into the cephalic vein once a day during 7 days.

The assessment of the animals was made on the basis of clinical signs, biological analyses carried out on the blood and the urine and of histopathological examinations.

These studies showed that the Pd-Bpheide a and its injectable Formulation 2 were well tolerated, provided the injections were made slowly, so that the oligomers present could disaggregate progressively into monomers.

Injectable Formulation 3

Formulation 3 containing 5 mg/ml of Pd-Bpheide a as photosensitising agent

Carrier:

Organic phase: benzyl alcohol/ethanol (20/80) 5% vol. wt.

Surfactant: Solutol HS 15, 10% wt./wt.

NaOH: 0.028 M

pH adjusted to 7.2±0.4 with citric acid 0.01 M

Formulation 3 was prepared as follows:

In an appropriately sized glass container containing 5 mg of Pd-Bpheide a prepared as describe above, 50 μl of a 20:80 vol./vol. mixture of benzyl alcohol and of ethanol were added and the mixture was stirred moderately. Then, 100 mg of Solutol HS 15 10% wt./wt. were added to the mixture and the mixture was stirred with a “Vortex”. Thereafter, 500 μl of a sodium hydroxide solution NaOH 0.028 M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations (WIPP).

The exact composition of Formulation 3 is given in Table 3 hereafter:

TABLE 3

| Name of | Formula | | | Relevant |
|---|---|---|---|---|
| component | Per unit dose | Percentage (%) | Function | standards |

Photosensitising agent

| | | | |
|---|---|---|---|
| Pd-Bpheide a | 5 mg | 0.5% wt./wt. | AP |

Other components

| | | | | |
|---|---|---|---|---|
| Alcohol, absolute | 40 μl | 4% vol./wt. | Co-solvent | EP 4^{th} edition 1318, A2000 |
| Benzyl alcohol | 10 μl | 1% vol./wt. | Co-solvent | EP 4^{th} edition 0256, 1997 |
| Solutol HS 15 | 100 mg | 10% wt./wt. | Surfactant | |
| NaOH 0.028 M | 500 μl | 50% vol./wt. | Neutralising | EP 4^{th} edition 0677, 1997 |
| | | | agent | |
| Citric acid, | QS to | QS to | Buffering | EP 4^{th} edition 0456, 1997 |
| 0.01 M | 7.2 ± 0.4 | 7.2 ± 0.4 | agent | |
| WIPP | QS to 1 g | QS to 100 | Diluent | EP 4^{th} edition 0169, A2000 |

This Formulation 3 of the present invention is clear but strongly coloured (dark purple) and it does not contain any trace of a precipitate, which indicates that the Pd-Bpheide a is completely solubilized.

The UV absorption spectrum of the Pd-Bpheide a in this Formulation 3 of the present invention exhibited 5 main peaks at 344, 385, 538, 763 and 818 nm, with a ratio OD 763/818 close to 1, indicating that the monomer determined spectrally amounted to 50% of the product.

Under the ICH conditions, this Formulation 3 is stable at 4° C. during at least 6 months, as verified by HPLC and UV analyses.

Injectable Formulation 4

Formulation 4 containing 1 mg/ml of the Pd-Bpheide a as photosensitising agent

Carrier:

Organic phase: benzyl alcohol/ethanol (20/80) 5% vol. wt.

Surfactant: Cremophore®D EL 5% wt./wt.

NaOH: 0.007 M

pH adjusted to 7.2±0.4 with citric acid 0.01 M

Formulation 4 was prepared as follows:

In an appropriately sized glass container containing 1 mg of Pd-Bpheide a prepared as describe above, 50 μl of a 20:80 vol./vol. mixture of benzyl alcohol and of ethanol were added and the mixture was stirred moderately. Then, 50 mg of Cremophore® EL were added to-the mixture and the mixture was stirred with a “Vortex”. Thereafter, 500 μl of a sodium hydroxide solution NaOH 0.007 M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with 0.01 M citric acid and the mixture was completed to 1 g with water for injectable preparations (WIPP).

The exact composition of Formulation 4 is given in Table 4 hereafter:

TABLE 4

| Name of | Formula | | | Relevant |
|---|---|---|---|---|
| component | Per unit dose | Percentage (%) | Function | standards |

Photosensitising agent

| | | | |
|---|---|---|---|
| Pd-Bpheide a | 1 mg | 0.1% wt./wt. | AP |

Other components

| | | | | |
|---|---|---|---|---|
| Alcohol, absolute | 40 μl | 4% vol./wt. | Co-solvent | EP 4^{th} edition 1318, A2000 |
| Benzyl alcohol | 10 μl | 1% vol./wt. | Co-solvent | EP 4^{th} edition 0256, 1997 |
| Cremophore ® | 50 mg | 5% wt./wt. | Surfactant | EP 4^{th} edition 1082, 1997 |
| EL | | | | |
| NaOH 0.007 M | 500 μl | 50% vol./wt. | Neutralising | EP 4^{th} edition 0677, 1997 |
| | | | agent | |
| Citric acid, | QS to | QS to | Buffering | EP 4^{th} edition 0456, 1997 |
| 0.01 M | 7.2 ± 0.4 | 7.2 ± 0.4 | agent | |
| WIPP | QS to 1 g | QS to 100 | Diluent | EP 4^{th} edition 0169, A2000 |

This Formulation 4 of the present invention is clear but strongly coloured (dark purple) and it does not contain any trace of a precipitate, which indicates that the Pd-Bpheide a is completely solubilized.

The UV absorption spectrum of the Pd-Bpheide a in the formulation 4 of the present invention exhibited 5 main peaks at 344, 385, 538, 763 and 818 nm with a ratio OD 763/818 of 80/20, indicating that the monomer determined spectrally amounted to 80% of the product.

Under the ICH conditions, this Formulation 4 is stable at 4° C. during at least 6 months, as verified by HPLC and UV analyses.

Injectable Formulation 5

Formulation 5 containing 1 mg/ml of Pd-Bpheide a as the photosensitising agent

Carrier in the aqueous phase:

Organic phase: propylene glycol 5% vol./wt.

Surfactant: Cremophore® EL 5% wt./wt.

NaOH: 0.007 M

pH adjusted to 7.2±0.4 with citric acid 0.01 M

Formulation 5 was prepared as follows:

In an appropriately sized glass container containing 1 mg of Pd-Bpheide a prepared as describe above, 50 μl of propylene glycol were added and the mixture was stirred moderately. Then, 50 mg of Cremophore® EL were added to the mixture and the mixture was stirred with a “Vortex”. Thereafter, 500 μl of a sodium hydroxide solution NaOH 0.007 M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations-(WIPP).

The exact composition of Formulation 5 is given in Table 5 hereafter:

TABLE 5

| Name of | Formula | | | Relevant |
|---|---|---|---|---|
| component | Per unit dose | Percentage (%) | Function | standards |

Photosensitising agent

| | | | |
|---|---|---|---|
| Pd-Bpheide a | 1 mg | 0.25% wt./wt. | AP |

Other components

| | | | | |
|---|---|---|---|---|
| Propylene glycol | 50 μl | 5% vol./wt. | Co-solvent | EP 4^{th} edition 0430, 1997 |
| Cremophore ® | 50 mg | 5% wt./wt. | Surfactant | EP 4^{th} edition 1082, 1997 |
| EL | | | | |
| NaOH 0.007 M | 500 μl | 50% vol./wt. | Neutralising | EP 4^{th} edition 0677, 1997 |
| | | | agent | |
| Citric acid, | QS to | QS to | Buffering | EP 4^{th} edition 0456, 1997 |
| 0.01 M | 7.2 ± 0.4 | 7.2 ± 0.4 | agent | |
| WIPP | QS to 1 g | QS to 100 | Diluent | EP 4^{th} edition 0169, A2000 |

This Formulation 5 of the present invention is clear but strongly coloured (dark purple) and it does not contain any trace of a precipitate, which indicates that the Pd-Bpheide a is completely solubilized.

The UV absorption spectrum of the Pd-Bpheide a in this formulation 5 of the present invention exhibited 5 main peaks at 344, 385, 538, 763 and 818 nm with a ratio OD 763/818 of 60/40, indicating that the monomer determined spectrally amounted to 60% of the product.

Under the ICH conditions, this Formulation 5 is stable at 4° C. during at least 6 months, as verified by HPLC and UV analyses.

Comparative Formulation 1

Comparative Formulation 1 containing 1 mg/ml of Pd-Bpheide a

Carrier:

Surfactant: Cremophore® EL 5% wt./wt.

NaOH: 0.007 M

pH adjusted to 7.2±0.4 with citric acid 0.01 M

The Comparative Formulation 1 was prepared as follows:

In an appropriately sized glass container containing 1 mg of Pd-Bpheide a prepared as describe above, 50 mg of Cremophore® EL were added and the mixture was stirred with a “Vortex”. Thereafter, 500 μl of a sodium hydroxide solution NaOH 0.007 M were added to the mixture and the mixture was stirred a few seconds with a “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations (WIPP).

The exact composition of the Comparative Formulation 1 is given in Table 6 hereafter:

TABLE 6

| | Formula | | | |
|---|---|---|---|---|
| Name of | Per | Percentage | | Relevant |
| component | unit dose | (%) | Function | standards |
| Pd-Bpheide a | 1 mg | 0.1% wt./wt. | AP | |
| Cremophore ® | 50 mg | 5% wt./wt. | Surfactant | EP 4^{th} edition |
| EL | | | | 1082, 1997 |
| NaOH 0.007 M | 500 μl | 50% vol./wt. | Neutralising | EP 4^{th} edition |
| | | | agent | 0677, 1997 |
| Citric acid, | QS to | QS to | Buffering | EP 4^{th} edition |
| 0.01 M | 7.2 ± 0.4 | 7.2 ± 0.4 | agent | 0456, 1997 |
| WIPP | QS to 1 g | QS to 100 | Diluent | EP 4^{th} edition |
| | | | | 0169, A2000 |

This Comparative Formulation 1 contains a precipitate, which demonstrates that the Pd-Bpheide a is not completely solubilized, in the absence of the benzyl alcohol/ethanol mixture or of propylene glycol.

Comparative Formulation 2

Comparative Formulation 2 containing 1 mg/ml of Pd-Bpheide a

Carrier:

Ethanol 5%

Surfactant: Cremophore® EL 5% wt./wt.

NaOH: 0.007 M

pH adjusted to 7.2±0.4

The Comparative Formulation 2 was prepared as follows:

In an appropriately sized glass container containing 1 mg of Pd-Bpheide a prepared as describe above, 50 μl of ethanol were added and the mixture was stirred moderately. Then, 50 mg of Cremophore® EL were added and the mixture was stirred with a “Vortex”. Thereafter, 500 μl of a sodium hydroxide solution NaOH 0.007 M were added to the mixture and the mixture was stirred a few seconds with the “Vortex”. The mixture was then subjected to ultrasounds during 30 minutes (temperature <25° C.) while stirring from time to time manually. Then, the pH was adjusted to 7.2±0.4 with citric acid 0.01 M and the mixture was completed to 1 g with water for injectable preparations (WIPP).

The exact composition of the Comparative Formulation 2 is given in Table 7 hereafter:

TABLE 7

| | Formula | | | |
|---|---|---|---|---|
| Name of | Per | Percentage | | Relevant |
| component | unit dose | (%) | Function | standards |
| Pd-Bpheide a | 1 mg | 0.1% wt./wt. | AP | |
| Ethanol | 50 μl | 5% vol./wt. | Co-solvent | EP 4^{th} edition |
| | | | | 1318, A 2000 |
| Cremophore ® | 50 mg | 5% wt./wt. | Surfactant | EP 4^{th} edition |
| EL | | | | 1082, 1997 |
| NaOH 0.007 M | 500 μl | 50% vol./wt. | Neutralising | EP 4^{th} edition |
| | | | agent | 0677, 1997 |
| Citric acid, | QS to | QS to | Buffering | EP 4^{th} edition |
| 0.01 M | 7.2 ± 0.4 | 7.2 ± 0.4 | agent | 0456, 1997 |
| WIPP | QS to 1 g | QS to 100 | Diluent | EP 4^{th} edition |
| | | | | 0169, A2000 |

This Comparative Formulation 2 flocculates, which proves that the Pd-Bpheide a is not completely solubilized, in the absence of benzyl alcohol.

1. An injectable galenic formulation for use in a diagnosis or in a photodynamic therapy (PDT), said galenic formulation containing:
a compound represented by the following general formula (I): 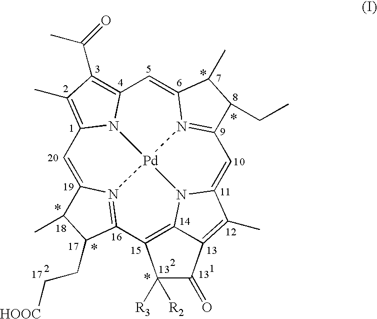
in which
R₂ represents a group H, OH or COOR₄, wherein R₄ is a hydrogen or a C₁-C₁₂ alkyl or a C₃-C₁₂ cycloalkyl,
R₃ represents H, OH or a C₁-C₁₂ alkyl or alcoxy and
* represents an asymmetric carbon,
in the form of a salt of an alkaline metal, in an amount not exceeding 10 mg/ml, as photosensitising agent, and
a pharmaceutically acceptable carrier in an aqueous phase,
characterised in that the pharmaceutically acceptable carrier in an aqueous phase contains at least a mixture of benzyl alcohol—ethanol in a ratio from 15:85 to 25:75 or propylene glycol, acting as a solubilizing agent for the photosensitising agent and a surfactant capable of decreasing the aggregation of the photosensitising agent, said surface-active agent being contained in an amount not exceeding 20 wt. % relative to the total weight of the formulation.

2. A injectable formulation according to claim 1, characterised in that the photosensitising agent of Formula (I) is palladium-bacteriopheophorbide a (Pd-Bpheide a) represented by the following Formula (II): 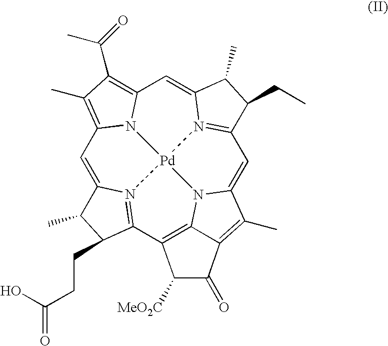
in the form of a salt of an alkaline metal.

3. A galenic formulation according to claim 1 or 2, characterised in that the compound of Formula (I) or the compound of Formula (II) is in the form of the sodium salt.

4. An injectable formulation according to any one of claims to 3, characterised in that the solubilizing agent is a mixture of benzyl alcohol—ethanol contained in the formulation in an amount not exceeding 5 vol. % relative to the total weight of the formulation.

5. An injectable galenic formulation according to any one of claims 1 to 4, characterised in that the solubilizing agent is a mixture of benzyl alcohol—ethanol in a ratio 20:80 vol./vol.

6. An injectable formulation according to any one of claims 1 to 3, characterised in that the solubilizing agent is propylene glycol, contained in the formulation in an amount not exceeding 5 vol. % relative to the total weight of the formulation.

7. An injectable formulation according to any one of claims 1 to 6, characterised in that the surface-active agent is Cremophore® EL, Cremophore® EL P or Solutol HS 15™.

8. An injectable galenic formulation according to any one of claims 1 to 7, characterised in that it contains, as the surface-active agent, Cremophore® EL or Cremophore® EL P in an amount not exceeding 5 wt. % relative to the total weight of the formulation.

9. A method for the preparation of an injectable galenic formulation for use in a diagnosis or in a photodynamic therapy (PDT) containing a compound represented by the following Formula (I): 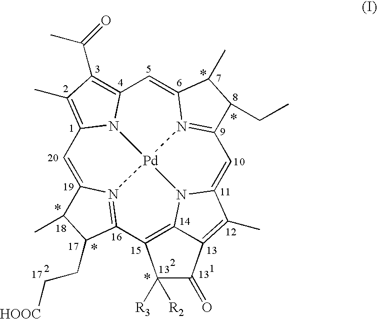
in which
R₂ represents a group H, OH or COOR₄, wherein R₄ is a hydrogen or a C₁-C₁₂ alkyl or a C₃-C₁₂ cycloalkyl,
R₃ represents H, OH or a C₁-C₁₂ alkyl or alcoxy and represents an asymmetric carbon,
in the form of a salt of an alkaline metal, in a amount of up to 10 mg/ml, as the photosensitising agent and a pharmaceutically acceptable carrier in an aqueous phase, said method including, in the order indicated, the steps consisting in:
(1) wetting the photosensitising agent of Formula (I) in its acid form with a solubilizing agent comprised of a mixture of benzyl alcohol—ethanol in a ratio from 15:85 to 25:75, or of propylene glycol;
(2) adding a surface-active agent in an amount not exceeding 20 wt. % relative to the final weight of the formulation
(3) adding a hydroxide of an alkaline metal in an amount sufficient for neutralising the acid function of the photosensitising agent of Formula (I);
(4) stirring the mixture in the aqueous phase sufficiently to ensure a complete conversion of the photosensitising agent into the salt thereof and its complete solubilization;
(5) adding a buffering agent designed for adjusting the pH to a physiologically acceptable value of 7.2±0.4;
(6) if necessary, adjusting the volume of the formulation with water for injectable preparations.

10. A method for the preparation of an injectable galenic formulation according to claim 9, characterised in that the photosensitising agent of Formula (I) in its acid form is palladium-bacteriopheorphorbide a (Pd-Bpheide a) represented by the following Formula (II):

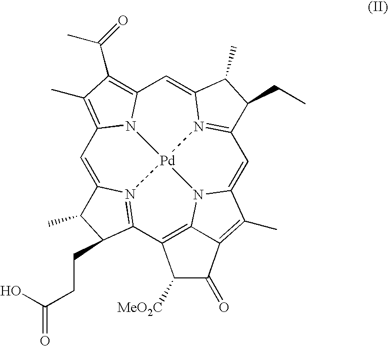

11. A method for the preparation of an injectable galenic formulation according to claim 9 or 10, characterised in the solubilizing agent is added in an amount not exceeding 5 vol. % relative to the total weight of the solution.

12. A method for the preparation of an injectable galenic formulation according to any one of claims 9 to 11, characterised in that the solubilizing agent added is a mixture of benzyl alcohol—ethanol in a ratio of 20:80.

13. A method for the preparation of an injectable galenic formulation according to any one of claims 9 to 12, characterised in that the surface-active agent added is Cremophore® EL or Cremophore® EL P or Solutol HS 15™.

14. A method for the preparation of an injectable galenic formulation according to any one of claims 9 to 13, characterised in that the surface-active agent added is Cremophore® EL or Cremophore® EL P and in that it is added in an amount not exceeding 5 wt. % relative to the total weight of the formulation.

15. A method for the preparation of an injectable galenic formulation according to any one of claims 9 to 14, characterised in that the hydroxide of an alkaline metal is sodium hydroxide.

16. A method for the preparation of an injectable galenic formulation according to any one of claims 9 to 15, characterised in that the buffering agent designed for adjusting the pH, which is added, is citric acid.

17. A method for the preparation of an injectable galenic formulation according to any one of claims 9 to 16, characterised in that the steps (1) to (6) are carried out under an atmosphere of an inert gas.

18. An injectable galenic formulation obtained by the method according to any one of claims 9 to 17.
